(19) United States
(12) Patent Application Publication
(10) Pub. No.: US 2008/0243080 A1
(43) Pub. Date: Oct. 2, 2008

(54) Method and apparatus for vascular access

(76) Inventor: David W. Chang, Cupertino, CA (US)

Correspondence Address: DAVID CHANG, 10175 Danube Drive, Cupertino, CA, 95014, US

(21) Appl. No.: 12/072,683

(22) Filed: May 16, 2008

Related U.S. Application Data
(60) Provisional application No. 60/920,892, filed on Mar. 31, 2007.
(60) Provisional application No. 60/920,153, filed on Mar. 27, 2007.

(51) Int. Cl. A61M 5/178; A61B 17/08; A61M 29/00

(52) U.S. Cl. 604/164.01; 606/213; 606/200

(57) ABSTRACT

A method and apparatus is described that facilitates appropriate placement of a closure device and allows ease of placement with repeated access without bleeding. A suitable biocompatible polymer diaphragm such as silicone or nipple is place on the adventitia of the vessel through a small cutdown incision. The puncture surface is a diaphragm that functions as a self-sealing valve. For embodiments designed for small catheter access such as 4 to 9 Fr, this may be a polymer that elastically seals around the puncture once the catheter is removed. Especially for larger access catheters, such as 10 French to 24 French, a preformed aperture or slit may be cut into the material, allowing controlled and atraumatic penetration of the device and artery. The sealing element may also be fixed to the outside wall of the vessel with holdfasts through a method that facilitates percutaneous delivery through the use of a guiding wire. A method and apparatus is also described that minimizes the potential migration of emboli generated upstream from the access site, an anchoring cannula and method for facilitating localizing of the access device.

### BACKGROUND OF THE INVENTION

This invention is related to methods and apparatus for facilitating arterial access using an holdfast anchored diaphragm on the adventitial surface of the artery and an semi-permeable sheath with a filter segment to prevent potential embolization.

Access to the arterial circulation can be both diagnostic and therapeutic yet arterial bleeding, occlusion and embolization complicate such procedures. Currently, the standard access site for arterial procedures is the common femoral artery. Unfortunately, despite its anatomic accessibility, it is often the distal end of one of the most diseased and tortuous arterial segments in the body. This results in difficulty delivering treatment to diseases of the aorta and its branches, especially if large caliber endovascular devices are needed. In contrast, the common carotid artery is of similar caliber but is one of the least diseased arteries in a region at low risk for infection. Moreover, the anatomic accessibility of this artery puts it in much closer proximity to the brain and the heart, two of the most important arterial beds. In addition, in contrast to transfemoral access, it facilitates access to the arteries to the viscera, kidneys and other organs because of their natural downward angulation as well as access to the arteries of both legs from a single site. Furthermore, this apparatus can be adapted for central venous access to facilitate hemodialysis.

The prior art teaches sealing of a significant sized arterial puncture site (6 French or greater) using a closure device with sutures, plugs or clips applied at the time of procedure. Examples include operative placement of suture or percutanous implantation of a closure device (Perclose, angioseal, starclose). These closure systems only permit single use access to the artery. Furthermore, the percutanous devices to date have a failure rate of approximately 5 percent largely as a result inappropriate placement of the device on the artery which can be caused by translumenal access in a diseased artery.

The method of percutanous non extremity vascular access has not been adopted because of potential concerns about bleeding, occlusion and embolization. These risk of these complications increases with frequently necessary repeat procedures. Described here are methods and apparatus that minimize these complications of arterial access and therefore facilitate accessing preferred sites such as the carotid, subclavian or femoral artery.

When the vessel acts as the chamber, rapid flow within the vessel as well as its endothelial lining discourages thrombosis. The autologous tissue also resists infection. The method of creating such a device involves attaching a diaphragm or nipple that has a sealing function to the adventitia of the vessel by means of sutures, penetrating or scaffolding elements, tissue adhesives or some combination of the above. The diaphragm or nipple may also have an undersurface that facilitates tissue incorporation with a thin porous or textured biocompatible layer that anchors the device to the artery. Creating a chamberless access device is enabled by placement of the device on a suitably large access vessel, for example, the carotid, subclavian femoral, axillary, brachial artery or the jugular, axillary, subclavian or femoral vein.

Of particular utility is the subclavian artery that is large vessel that can be made ergonomically accessible, rarely diseased and located in a low infection risk area. An obstacle for convenient use of this vessel is the possibility of bleeding, leading to brachial plexus nerve injury, hematoma or hemathorax. In addition, inexperienced attempts to access this vessel may result in pneumothorax requiring chest tube decompression.

### BRIEF SUMMARY OF THE INVENTION

Disclosed here are methods and apparatus for chamberless vascular access and for facilitating access. The objective of eliminating the chamber of a vascular access device is achieved by making the vessel itself the chamber and incorporating the device as part the artery. The objective of facilitating access include methods and devices for automating the access process and preventing embolization.

### BRIEF DESCRIPTION OF THE DRAWING(S)

FIG. 1 is a method for vascular access that involves placement of a self sealing diaphragm or nipple on a vessel such as the supraclavicular or infraclavicular subclavian artery.

### DESCRIPTION OF THE INVENTION

Disclosed here are methods and apparatus for chamberless vascular access and for facilitating access. The objective of eliminating the chamber of a vascular access device is achieved by making the vessel itself the chamber and incorporating the device as part the artery. The objective of facilitating access include methods and devices for automating the access process and preventing embolization.

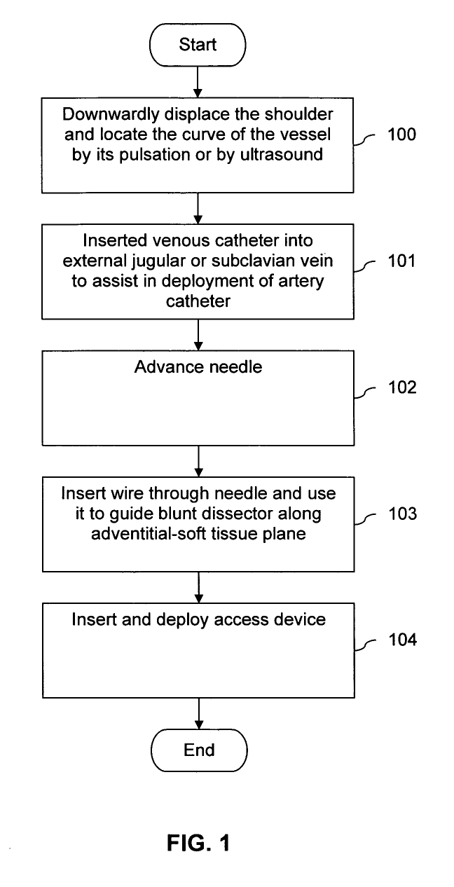

Described therein in FIG. 1 is a method for vascular access that involves placement of a self sealing diaphragm or nipple on a vessel such as the supraclavicular or infraclavicular subclavian artery. The supraclavicular portion of the vessel can be accessed most readily by downward displacement of the shoulder, locating the curve of the vessel by its pulsation or by ultrasound (100). Further landmarks for a large individual include the mid clavicle and the external jugular vein as it courses to the clavicle. A venous catheter as described below may be inserted into the external jugular or the subclavian vein to assist in deployment of the artery catheter (101). In one embodiment of the method, 22-18 gauge needle is advanced into the artery parallel to the clavicle, avoiding possibility of puncture of the lung. In other, the needle with or without ultrasound guide is advanced as a steeper angle but no more the level of the artery as determined by palpation or imaging study. (102) A wire is inserted through the needle and used to guide a blunt dissector along the adventitial-soft tissue plane (103). The access device is then inserted and deployed (104). Alternatively, open surgical exposure of the artery is performed and the diaphragm or nipple attached to the vessel.

A method and apparatus is described that facilitates appropriate placement of the closure device and allows ease of placement with repeated access without bleeding. A suitable biocompatible polymer diaphragm such as silicone is place on the adventitia of the vessel through a small cutdown incision. The puncture surface is a diaphragm that functions as a self-sealing valve. For embodiments designed for small catheter access such as 4 to 9 Fr, this may be a polymer that elastically seals around the puncture once the catheter is removed. For larger access catheters, such as 10 French to 24 French, a preformed aperture or slit(s) cut into the material, allowing controlled atraumatic penetration of the device and artery. The sealing element may also be fixed to the wall of the vessel with holdfasts through a method that facilitates percutaneous delivery through the use of a guiding wire.

The diaphragm is designed to have arc of 60 to 180 degrees with a diameter between 90 to 100% of the arterial diameter. Flexibility and some appropriate directional rigidity can be achieved by reinforcing the diaphragm with a wire frame made of metal such nitinol. For the carotid or femoral artery, holdfasts include tissue glue, collar elements that may be 6 to 9 mm diameter (but need not be circumferential around the artery) or embedded holdfasts that penetrate the wall of the artery but does not obstruct the lumen. These may include sharp tines with the proximal end embedded in the diaphragm with process known in the art. or may be of one body with the frame or collar. The distal sharp end embeds in the wall of the artery at an angle from 5 degrees to 60 degrees so as to fix the diaphragm to the artery or up to 90 degrees with the use of self locking tines. The tines also prevent vessel tissue from prolapsing into the lumen after removal of large shealths (>10 French). Fixation is achieved with counter angled tines or parallel matrix of tines in an arc configurations with anchoring tines. The tines can be angled so that anchoring is achieved in a direction opposite to entry. This allow ease of movement of the diaphragm edge which can acts as a dissecting tool to create space in the vessel sheath

The advantages of the invention include ability to perform a minimally invasive access implant, minimal thrombotic risk because translumenal fixation is not necessary, large target area for repeated access, low intravascular infection risk and broad applicability to wide range of sheath sizes.

Another method and apparatus is described that minimizes the potential migration of emboli generated upstream from the access site. A sheath is designed to enable a portion of the sheath wall to function as a filter. The wall may be constructed with a porous section made of polymer fiber or laser drilled holes in metal or polymer. Alternatively, a filter recessed between a outer and inner wall of the sheath tip with blood exiting a side opening(s) downstream from an occluding element can be used. A retractable anchor such as a non-occluding balloon or protrusion is placed on the sheath downstream of the filter section to prevent dislodgement of the filter into the extravascular space. An occlusive balloon may also be placed on the sheath upstream from the filter section to divert any emboli from unimpended downstream movement.

There exists an artery 1 with an introducing sheath 9 to 24 french. 2 The sheath may be introduced with the aid of ultrasound localization or manual palpation. A wire guide 3 may be inserted into the vessel to localize it or placed on it until transmitted pulsations are noted. The dilator 4 may have a rail 5 for the wire and is passed alongside the adventitia of the vessel. The rail has a split line so that the dilator can continue to glide along the adventitia distal to the insertion site of the wire. The diaphragm 6 may have a frame with crossbars 7 to create an appropriate arc for the vessel without excess mass. A glue tube 8 fastens to the underside of a diaphragm delivered rolled up in the sheath. Once the diaphragm is placed along the vessel, the glue tube with side holes applies the tissue glue and may be removed as pressure is exerted on the neck or groin.

There exists the same artery with a similar diaphragm and frame. Tines are embedded in the diaphragm angled away from the direction of insertion (for example, if done in a minimally invasive fashion) or in the same direction of insertion. A stiff rod 9 continuous with rigid spine(s) 10 housed with several guides 11 in the diaphragm. Once the diaphragm is delivered into the vessel sheath, the rod is pulled in the same direction as the tines while pressure is held on the neck or groin, seating the diaphragm to the vessel. The rod can be used to test the security of the attachment. If satisfactory, one way self locking anchoring counter tines 12 may be embedded in the artery wall with the aid of a pusher. The rod is removed. Alternatively, the device can be seated with pressure orthogonal to the vessel axis using self locking tines and or tissue glue.

Another embodiment has radially oriented tines with collar elements as holdfasts.

There exists the same artery with an applied diaphragm closure device and a sheath with an occluding element distally and filter segment proximally.

There exists an embolization protection sheath.

The device may have one or more nipple that is a chamberless compartment in continuity with the vessel through a potential channel that is compressed by the elastic properties of a suitable polymer, for example, a rubber, silicone, plastic. Alternatively, the device material is sponge-like in its interior and may have an impermeable lining along the potential channel. In an addition embodiment, the device may be of two or more pieces that fit seamlessly together to occlude the potential channel with one or more pieces spring loaded. It is understood that any combination of the apparatus and methods described may be used. The device is fixed to the vessel by any combination of penetrating anchors, tissue adhesive, circumferential bands, or textured or porous material that allows cellular ingrowth or incorporation. Growth factors, matrix elements, or materials found in access catheter cuffs may be used to facilitate incorporation.

The end away from the artery as an opening that guides a needle or blunt trocar into the potential channel directed at the vessel, facilitated access. Once, the needle or trocar is removed, the nipple seals over the puncture site in the vessel. The access needle or trocar may have a lumen that allows wire access enable placement of a larger catheter or sheath. The exit of this lumen may be disposed at the end or side of the accessing instrument. The needle, trocar or shealth may have one or more surface features that mate with the access device, preventing further advancement thereby preventing inadvertent injury to the intima of the vessel. This lock can be retractable, allow fixation of the access cannula and preventing inadvertent disengagement of the catheter during intervention, monitoring or treatment.

Described also is a method for introduction of therapeutic devices, for example, stents, stentgrafts, valves, occlusion devices, athrectomy devices, thrombolytic devices, clot retrieval devices, angioplasty devices and cannulas through safe subclavian artery 2access. One new method for hemodialysis using this invention uses one artery with an access device, for example, the left subclavian artery for the venous cannula for hemodialysis and another, for example, the right subclavian artery for the arterial cannula. The returning cannula can also be placed in any other suitable vessel, for example, the jugular, subclavian, or femoral veins. This facilitates hemodialysis by dispensing with the need for a pump and allows return of detoxified blood to circulation without risk of stroke.

What is claimed is:

1. An implantable self sealing diaphragm consisting of a surface that conforms to an arc of a vessel covering a length of the vessel with mechanical or chemical holdfasts that secure the outer vessel surface to it.

2. An apparatus as in claim 1 where the punctured device has a prefabricated aperture, slit, or plurality thereof partially or fully through its depth to facilitate entry of the puncturing device.

3. An apparatus as in claim 1 delivered folded within a sheath.

4. An apparatus as in claim 1 having a frame composed of a shape memory material with sharp tines 5 to 90 degrees that penetrate the wall of the vessel.

5. An apparatus as in claim 1 with collar elements or sutures that fix the diaphragm to the vessel.

6. An apparatus as in claim 1 which includes tubular structure within or adjacent to the diaphragm with apertures that deliver a tissue glue.

7. An apparatus as in claim 1 which has a porous or textured surface that allows tissue incorporation.

8. An apparatus as in claim 1 with radio-opaque, echo, thermal or magnetically distinctive markers that enable precise placement of a puncture apparatus.

9-11. (canceled)

12. An apparatus for therapeutic vascular procedures consisting of a sheath comprised of an occluding upstream element around the sheath and a filter segment of one body with the sheath downstream of the occluding element and a retractable intravascular anchor downstream of the filter.

13. An apparatus of claim 12 with a balloon occluding element.

14. An apparatus as in claim 12 with a retractable foot or non occlusive balloon downstream from the occluding segment.

15-17. (canceled)

18. A puncture cannula comprising of a sharp inner tube or rod that tranverses an outer blunt or tapered trocar with side opening to be inserted in the intravascular space.

19. An apparatus as in claim 18 with a protrusion or plurality of protrusions that mates with complementary elements on a self sealing vessel based access device.

20. An apparatus as in claim 18 with retractable wire element that engages with the self sealing vessel based access device.
